(19) United States
(12) Patent Application Publication
(10) Pub. No.: US 2006/0129046 A1
(43) Pub. Date: Jun. 15, 2006

(54) Methods and devices for coupling a needle to an ultrasound device and guiding advancement of the needle

(75) Inventors: Richard J. Stevens, San Jose, CA (US); Lawrence S. Polayes, Morgan Hill, CA (US)

Correspondence Address: HOEKENDIJK & LYNCH, LLP, P.O. BOX 4787, BURLINGAME, CA, 94011-4787, US

(73) Assignee: SHEATHING TECHNOLOGIES, INC., Morgan Hill, CA (US)

(21) Appl. No.: 11/003,915

(22) Filed: Dec. 3, 2004

(51) Int. Cl. A61B 8/14

(52) U.S. Cl. 600/464; 604/116

(57) ABSTRACT

A needle guide kit is provided which includes a needle guide, a retainer and a tether. The needle guide is coupled to an ultrasound device either directly or via an adapter. The needle guide has an open v-shaped slot which can receive a number of different needle sizes. The retainer can be attached to the needle guide to form a needle channel between the needle guide and retainer. Each retainer corresponds to a different needle size whereas each of the different needle guides correspond to a different trajectory for advancing the needle. The kit may also include a tether which is coupled to one of the components by the user. The tether prevents loss of one or more of the components when the components are detached or decoupled when completing the procedure.

### BACKGROUND OF THE INVENTION

The present invention relates to methods and devices for coupling a needle to an ultrasound device and for guiding advancement of the needle. The present invention is particularly adapted for use in biopsy procedures using a hand-held ultrasound device.

### SUMMARY OF THE INVENTION

In one aspect of the present invention, a kit for coupling a needle to an ultrasound device and guiding advancement of the needle is provided. The kit has a needle guide and a retainer. The needle guide defines the trajectory of the needle based upon the desired path or desired depth of penetration relative to the ultrasound device. The retainer is coupled to the needle guide to form a needle channel which receives and guides the needle.

The kit may include a number of different retainers with each retainer corresponding to a particular needle size. The needle guide may be designed to be used with a number of different needle sizes so that one needle guide may accommodate a number of different needle sizes when a different retainer is attached. To this end, the needle guide has a v-shaped slot which is relatively open to permit direct access and placement of the needle in the slot. The open nature of the slot permits direct removal or placement of the needle without requiring longitudinal movement of the needle relative to the needle guide.

In another aspect of the present invention, the kit may include a tether which maintains connection between the needle guide and the retainer. The tether is attached to one of the components, such as the retainer, and is then attached to the other component by the user. The tether is particularly useful in preventing loss of one of the components when the retainer is released from engagement or attachment to the needle guide. The tether may be retained or held in any suitable manner such as within a slot in the needle guide. The tether preferably remains coupled to both the needle guide and the retainer after detaching the retainer from the needle guide to prevent losing the retainer.

As mentioned above, the kit may have a number of different needle guides which are each adapted to guide the needle along a different path relative to the ultrasound device to which it is attached. The kit may also have a number of retainers to accommodate a number of different needle sizes when the desired trajectory (or depth) is known.

The needle guide may be coupled directly to the ultrasound device or may be coupled to an adapter which is attached to the ultrasound device. The ultrasound device may also be covered with a sheath to provide a sterile barrier with the adapter positioned either over or under the sheath. When the adapter is positioned over the sheath, the adapter may be used to secure the sheath to the ultrasound device.

These and other aspects of the invention will become apparent from the following description and drawings.

### BRIEF DESCRIPTION OF DRAWINGS

FIG. 1 shows a needle guide attached to an ultrasound device.

FIG. 2 shows a perspective view of the needle guide.

FIG. 3 shows another view of the needle guide.

FIG. 4 shows the needle guide prior to attachment to the ultrasound device.

FIG. 5 shows the needle guide attached to the ultrasound device.

FIG. 6 shows a retainer.

FIG. 7 shows the retainer prior to attachment to the needle guide.

FIG. 8 shows the retainer and a tether both attached to the needle guide.

FIG. 9 shows an adapter which is used to couple the needle guide to another ultrasound device.

FIG. 10 shows the adapter and needle guide prior to attachment to the ultrasound device.

FIG. 11 shows the adapter attached to the ultrasound device and the needle guide attached to the adapter.

FIG. 12 shows a sheath positioned over the adapter.

FIG. 13 shows the sheath positioned under the adapter.

FIG. 14 shows a needle coupled to and extending from the needle guide.

### DESCRIPTION OF THE PREFERRED EMBODIMENTS

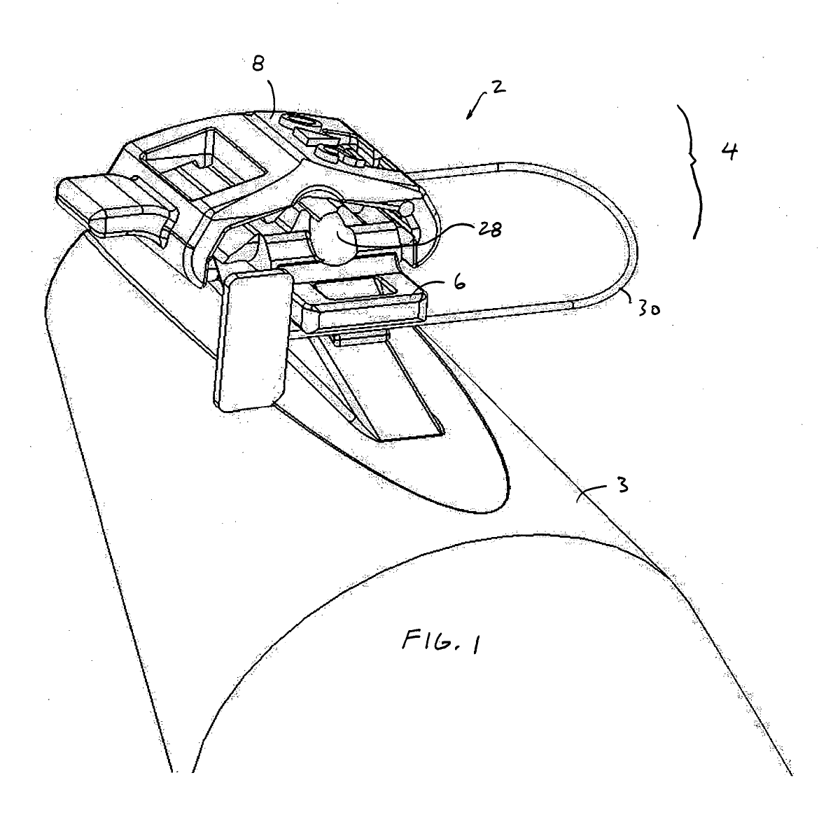

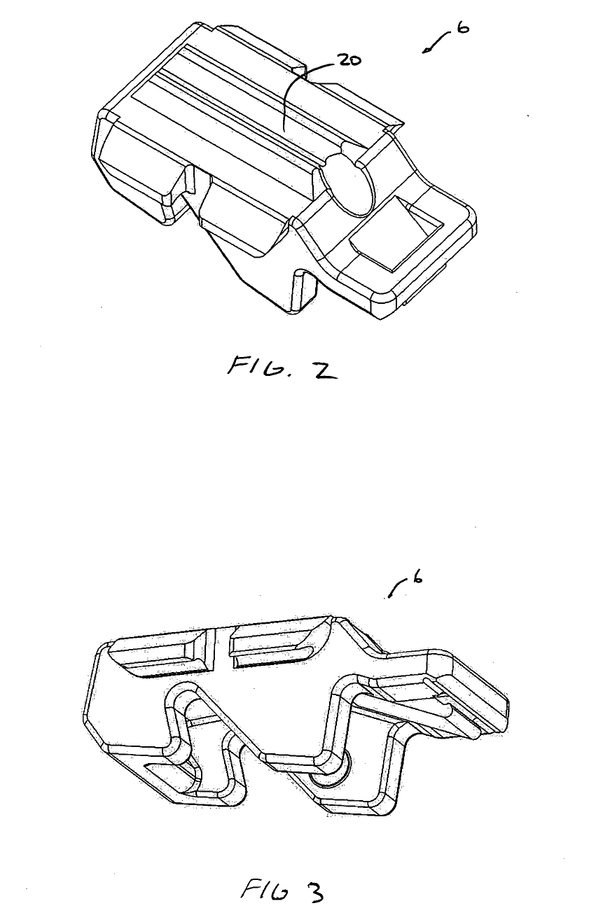

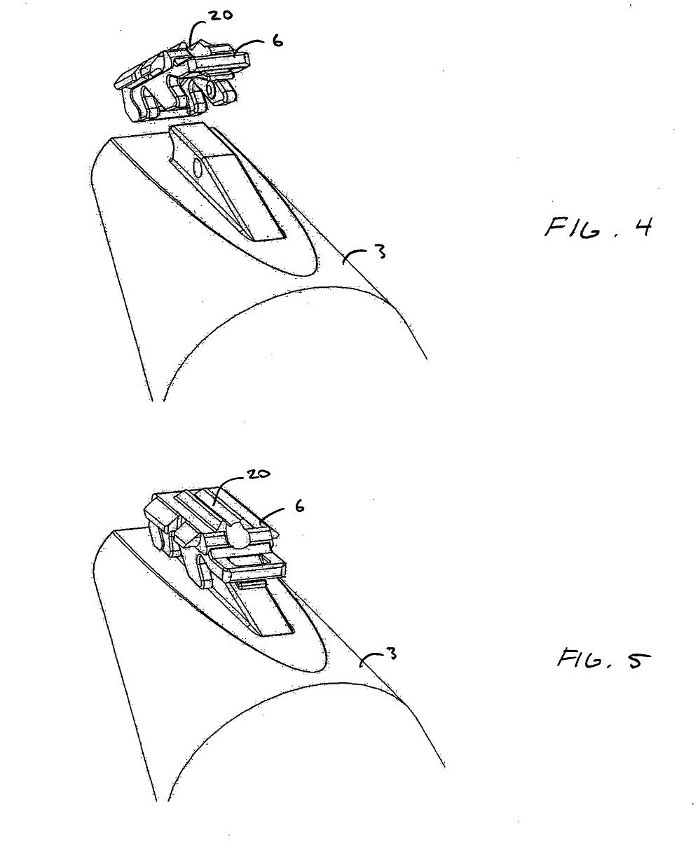

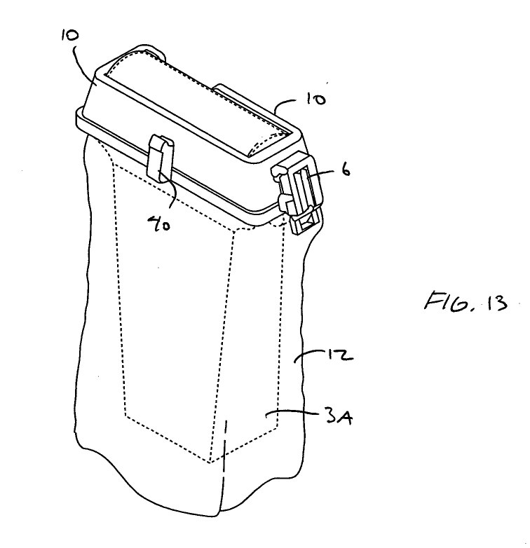

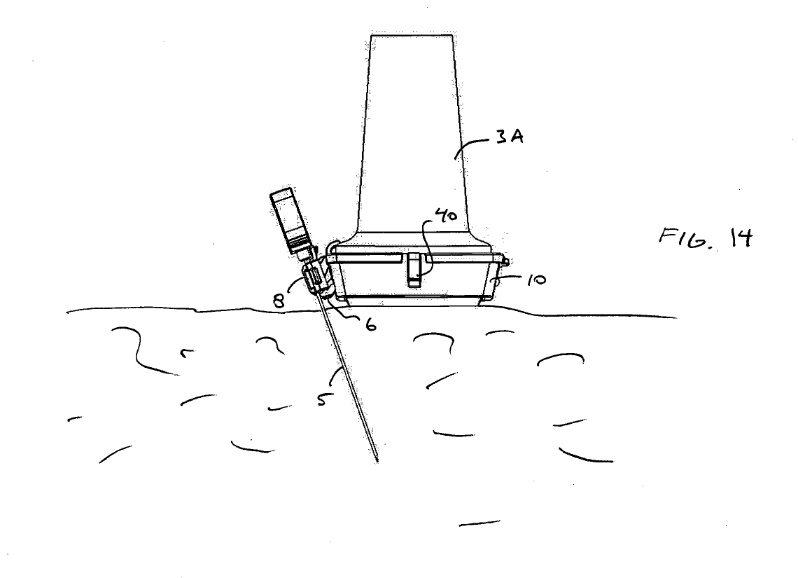

Referring to FIG. 1, a device 2 for coupling a needle to an ultrasound device 3 is shown. The device 2 may be provided as a kit 4 which is contained within a sterile pack (not shown) as will be described below. The kit 4 may include various components such as a needle guide 6 and a retainer 8. The kit 4 may also include an adapter 10 and/or a sheath 12 which are described below and shown in FIGS. 12 and 13. The needle (not shown) is held in a needle channel 28 formed between the needle guide 6 and the retainer 8. Referring to FIG. 14, a needle 5 is shown coupled to another ultrasound device 3A.

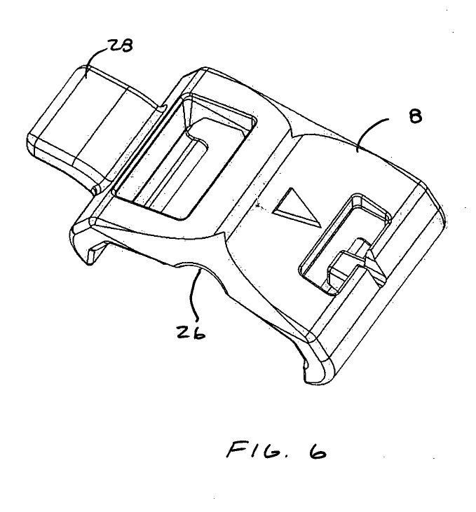

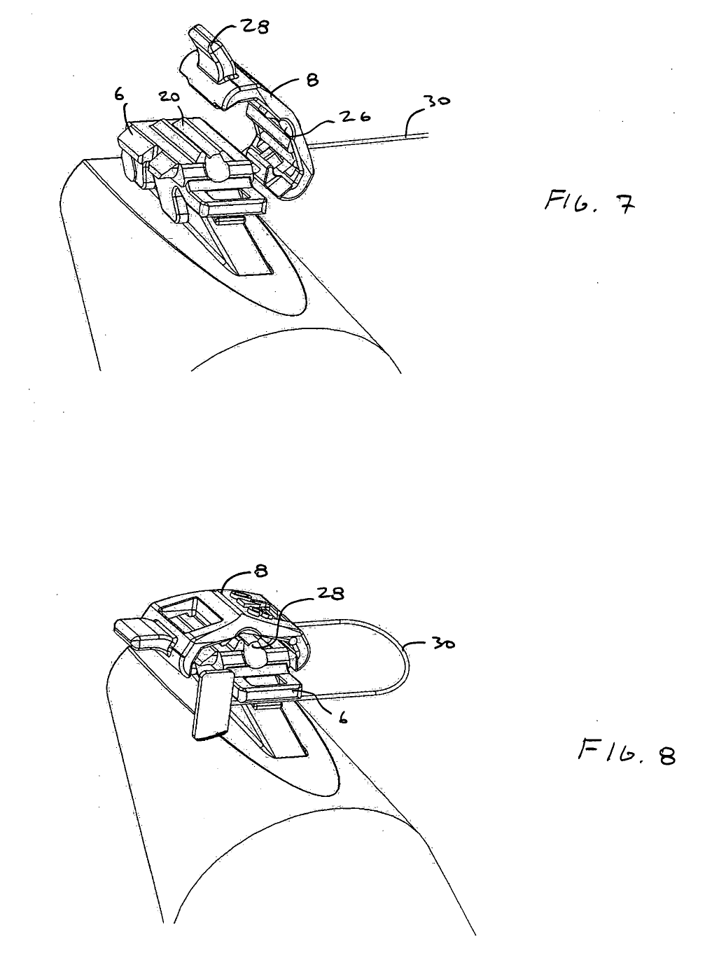

The needle guide 6 may be attached directly to the ultrasound device 3 as shown in FIG. 1 or may be coupled to the device 2 with the adapter 10 as shown in FIGS. 12 and 13. The needle guide 6 may be attached to the adapter 10 with the adapter 10 either over the sheath 12 (FIG. 13) or under the sheath 12 (FIG. 12). Stated another way, the needle guide 6 may be attached to the device 3 (or adapter 10) with the sheath 12 positioned over the device 3 (or adapter 10) as practiced in a number of known devices for guiding advancement of a needle. The needle guide 6 of the present invention is rotated into a snap fit engagement with the ultrasound device 2 as shown in FIGS. 4 and 5.

Referring still to FIG. 4, the needle guide 6 has a groove 20 on an upper side which forms part of the needle channel 28. The groove 20 permits the needle 5 to be placed directly into the guide 6 rather than directing the needle through a hole, slot or other structure as is sometimes necessary with other needle guiding devices. The groove 20 is U- or V-shaped and curved to accommodate different size needles as explained below. The needle guide 6 is configured to direct the needle 5 along a predetermined path or depth relative to the ultrasound device 3 as shown in FIG. 14.

One aspect of the present system is that the same needle guide 6 may be used with a number of different needle sizes. The relatively open nature of the groove 20 permits use of the same needle guide 6 with a number of different needle sizes with a different retainer 8 being used for each needle size. The groove 20 also permits direct placement or removal of the needle 5 without requiring relative longitudinal movement between the needle 5 and the needle guide 6 as is sometimes necessary with other needle guiding devices. Depending upon the particular application, the kit 4 may include a number of different retainers 8 with each retainer 8 corresponding to a different needle size. When the user determines the appropriate needle size for the procedure, the corresponding retainer 8 is selected and coupled to the needle guide 6 to form the appropriate size needle channel 28. The retainer 8 has a groove 26 which cooperates with the groove 20 in the needle guide 6 to form the needle channel 24.

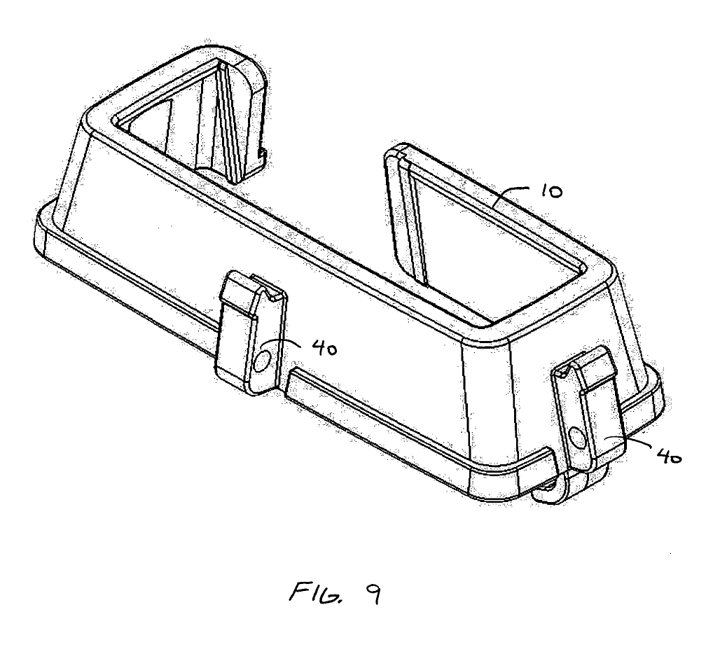

The retainer 8 has a snap fit connection with the needle guide 6 although any other suitable connection may be used. The retainer 8 is pivoted into a snap fit engagement with the needle guide 6 as shown in FIG. 7. When it is desired to release the needle, a release tab 28 is simply pulled to release the retainer 8 from the snap fit connection with the guide 6. After the retainer 8 has been released, the needle 5 may be easily removed due to the relatively open nature of the v-shaped groove 20. The retainer 8 may be attached to the needle guide 6 with the tab 28 facing in either direction depending upon user preference.

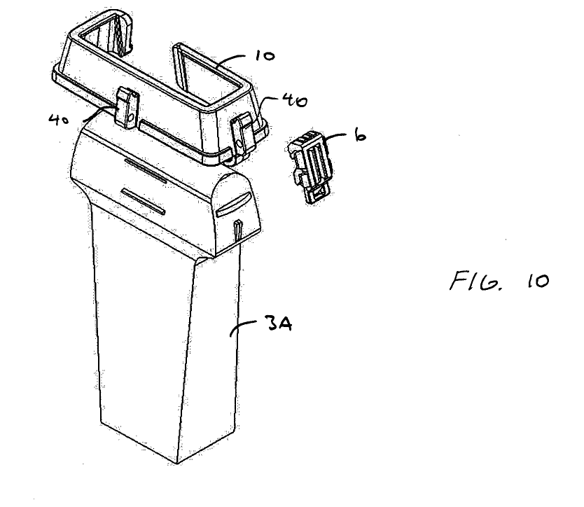

A tether 30 is used to maintain a connection between the retainer 8 and needle guide 6 after detaching the retainer 8 from the needle guide 6. The tether 30 is preferably attached to either the retainer 8 or the needle guide 6 and is then attached to the other component by the user. In FIG. 8, the tether 30 is attached to the retainer 8 and the user snaps part of the tether 30 into a slot 32 in the needle guide 6. Of course, the tether 30 may be releasably attached to the needle guide 6 in any other suitable manner. The tether 30 helps to prevent losing one of the parts when detaching the retainer 8 from the needle guide 6.

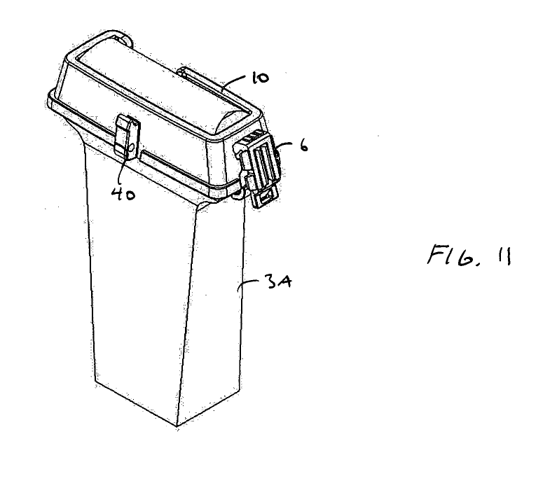

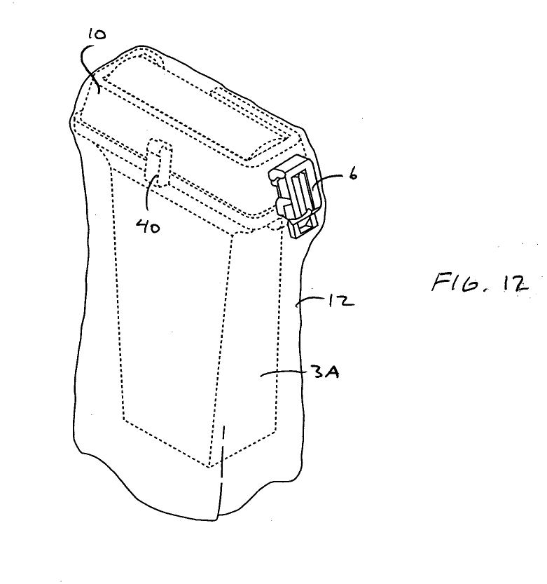

Referring now to FIGS. 9-14, the kit 4 may also include the adapter 10 which is coupled to an ultrasound device 3A. The adapter 10 is used to couple the same needle guide 6 to a different ultrasound device 3A. The adapter 10 may be reusable or may be disposable and provided with the kit 4. The needle guide 6 is attached to the adapter 10 as shown in FIGS. 12 and 13 either over or under the sheath 12. The adapter 10 may take many forms such as a generally G-shaped structure. The adapter 10 may be specially designed to fit the particular ultrasound device 3A being used or may fit several different ultrasound devices 3. It is understood that different adapters 10 may be required for different ultrasound devices 3, 3A. Of course, the needle guide 6 may also be attached directly to the ultrasound device 3 as shown in FIG. 1 without the use of an adapter 10.

The adapter 10 has up to four connections 40 which are each designed to receive the needle guide 6. The connections are oriented 90 degrees from one another so that the needle 5 may be guided in or across the image plane from either side of the ultrasound device 3 as desired. The appropriate needle guide 6 is then attached to the adapter 10 to guide the needle 5 along the desired trajectory or to the desired depth.

The sheath 12 may also be used to cover the ultrasound device 3A and provide a sterile barrier around the ultrasound device 3A. As mentioned above, the sheath 12 may be positioned over the adapter 10 as shown in FIG. 12 or under the adapter 10 as shown in FIG. 13. When the adapter 10 is positioned over the sheath 12 the adapter 10 may be used to secure the sheath 12 to the ultrasound device 3A.

One kit 4 which is contemplated by the present invention includes a number of different needle guides 6 which each represent a different depth or trajectory relative to the ultrasound device 3A. The kit 4 may include one retainer 8 which is suitable when the user knows the size of needle 5 required or the kit 4 may include a number of retainers 8 for different needle sizes when the needle size is not known in advance. An exemplary kit 4 may include 2-6 needle guides 6 and one retainer 8 when the needle size is known or 2-4 retainers to accommodate more needle sizes when the needle size is not known in advance. As mentioned above, the kit 4 may also include the adapter 10 and/or sheath 12 as well.

The present invention has been described in connection with preferred embodiments, however, it is understood that the invention may be practiced using other devices, methods and structures without departing from the scope of the invention. For example, the device has been described in connection with guiding and advancement of a needle but may be used to guide advancement of any other medical device such as an ablation probe. Furthermore, the needle may be used for any purpose such as extraction of tissue or fluid or injection of a fluid or substance, such as a marker, into the patient.

1. A method of coupling a needle to an ultrasound device and guiding advancement of the needle relative to the ultrasound device, comprising the steps of:
providing a kit which is used to couple a needle to an ultrasound device, the kit having a plurality of needle guides, wherein each needle guide is adapted to be coupled to the ultrasound transducer and each needle guide being configured to guide a needle along a different trajectory relative to the ultrasound transducer, the kit also having a retainer;
selecting a desired needle guide from the plurality of needle guides based upon a desired trajectory for the needle relative to the ultrasound device;
coupling the desired needle guide to the ultrasound device;
attaching the retainer to the desired needle guide, the retainer and the needle guide forming a needle channel; and
positioning a needle in the needle channel formed by the retainer and the desired needle guide.

2. The method of claim 1, wherein:
the providing step is carried out with the retainer configured to be coupled to each of the plurality of needle guides.

3. The method of claim 1, wherein:
the providing step is carried out with a plurality of retainers, wherein each of the retainers forms the needle channel with a different size.

4. The method of claim 1, further comprising the step of:
covering the ultrasound transducer with a sheath before the coupling step;
the coupling step being carried out with the desired needle guide being coupled to the ultrasound device with the sheath positioned between the ultrasound device and the desired needle guide.

5. The method of claim 1, wherein:
the providing step is carried out with the retainer having a tether which couples the retainer to the guide.

6. The method of claim 5, further comprising the step of:
attaching the tether to at least one of the desired needle guide and the retainer after the selecting step.

7. The method of claim 5, further comprising the step of:
the providing step is carried out with the tether being coupled to the retainer; and
the attaching step being carried out by attaching the tether to the needle guide.

8. The method of 7, wherein:
the attaching step is carried out with the tether being held in a slot on the needle guide.

9. The method of claim 1, wherein:
the providing step is carried out with the needle guide having an exposed groove, wherein the exposed groove is configured to receive a number of different needle sizes when the retainer is coupled thereto.

10. The method of claim 1, further comprising the step of:
connecting an adapter to the ultrasound transducer.

11. The method of claim 10, further comprising the step of:
covering the ultrasound transducer with a sheath after the connecting step and before the coupling step so that the adapter is positioned beneath the sheath and the needle guide is positioned outside the sheath.

12. The method of claim 10, further comprising the step of:
covering the ultrasound transducer with a sheath before the connecting step and before the coupling step so that the adapter and the needle guide are positioned outside the sheath.

13. A method of coupling a needle to an ultrasound device and guiding advancement of the needle, comprising the steps of:
providing a needle guide, a retainer and a tether;
coupling the needle guide to the ultrasound transducer;
attaching the retainer to the needle guide so that a needle channel is formed between the needle guide and the retainer;
positioning a needle in the needle channel; and
detaching the retainer from the needle guide while the tether remains coupled to both the needle guide and the retainer.

14. The method of claim 13, wherein:
the attaching step is carried out with a snap fit between the retainer and the needle guide.

15. The method of claim 13, wherein:
the providing step is carried out with the tether being attached to one of the needle guide and retainer before the coupling step.

16. The method of claim 15, further comprising the step of:
connecting the tether to the other one of the needle guide and retainer.

17. The method of claim 13, wherein:
the connecting step is carried out by coupling the tether to the needle guide.

18. The method of claim 13, wherein:
the providing step is carried out with the plurality of retainers, wherein each retainer corresponds to a different needle size.

19. The method of claim 18, further comprising the step of:
the coupling step is carried out by selecting one of the plurality of retainers.

20. The method of claim 13, wherein:
the attaching step is carried out by connecting an adapter to the ultrasound transducer and attaching the needle guide to the adapter.

21. The method of claim 13, further comprising the step of:
covering the ultrasound transducer with a sheath before the attaching step.

22. The method of claim 21, wherein:
the coupling step is carried out by coupling the needle guide to the adapter with the sheath positioned between the needle guide and the adapter.

23. The method of claim 21, wherein:
the coupling step is carried out by coupling the needle guide to the adapter with the sheath positioned between the adapter and the ultrasound transducer.

24-54. (canceled)
